(19) United States
(12) Patent Application Publication
(10) Pub. No.: US 2017/0340476 A1
(43) Pub. Date: Nov. 30, 2017

(54) ENHANCED INTRAUTERINE DEVICE

(71) Applicant: Martin Kuster, Walchwil (CH)

(72) Inventor: Martin Kuster, Walchwil (CH)

(21) Appl. No.: 15/605,650

(22) Filed: May 25, 2017

Related U.S. Application Data
(60) Provisional application No. 62/341,326, filed on May 25, 2016.

(51) Int. Cl. A61F 6/14; A61F 6/18; A61B 5/01; A61B 90/98; A61B 5/145; A61M 31/00; A61B 5/00

(52) U.S. Cl. CPC A61F 6/144; A61M 31/002; A61F 6/18; A61B 5/4839; A61B 5/002; A61B 5/0031; A61B 5/6875; A61B 90/98; A61B 5/14532; A61B 5/14503; A61B 5/01; A61B 2560/0219; A61B 2560/0481; A61B 2562/06

(57) ABSTRACT

The present disclosure relates to an enhanced intrauterine device. Some aspects may involve a body, at least one arm, at least one connector, a communication circuit, and a removal mechanism. The body may be inserted in a uterus. The at least one arm may in a first position extend radially from the body and in a second position be detached from the body. The at least one connector may link the at least one arm to the body such that the at least one arm remains linked to the body in both the first position and second position. The communication circuit may couple to at least one of the body and the at least one arm for responding to a first signal by transmitting a second signal. The removal mechanism may couple to body and may include a magnetic portion for removing the intrauterine device from the uterus.

### CROSS REFERENCE TO RELATED APPLICATIONS

This disclosure is related to and claims the benefit of priority of U.S. Provisional Application No. 62/341,326, titled “Enhanced Intrauterine Device” and filed on May 25, 2016, which is hereby incorporated by this reference in its entirety.

### TECHNICAL FIELD

The present disclosure relates to a female contraceptive device and, more particularly (although not necessarily exclusively), to an enhanced intrauterine device.

### BACKGROUND

An intrauterine device (“IUD”) may be inserted into a uterus to provide long-term contraception. IUDs may be made of metal, plastic, or other suitable substances in various shapes and sizes. An IUD can be inserted into the uterus through the cervix and the IUD may include arms that extend radially away from a body of the IUD (e.g., forming a “T” shape) after insertion.

Some IUDs include a substance that can be released into the uterus to prevent fertilization. For example, an IUD may be wrapped with copper wire such that it releases a small amount of copper into the uterus, which may work as a spermicide. In another example, an IUD may release a synthetic hormone (e.g., levonorgestrel) into the uterus that thickens the cervical mucus so that sperm cannot reach an egg. Additionally or alternatively, the IUD may cause inflammation in the uterus that prevents the egg from implanting on the uterine wall.

An IUD may provide contraception for years (e.g., a hormone IUD may provide five years of contraception and a copper IUD may provide ten years of contraception). But, the IUD can be pushed through the cervix and even expelled completely from the uterus without the user noticing. Thus, an IUD user may periodically check for the presence and proper placement of the IUD in the uterus.

Some IUDs include removal strings that extend from the body of the IUD in the uterus, through the cervix, and into the vagina. The presence and proper placement of an IUD can be determined by locating the removal strings. To remove the IUD, the removal strings may be grasped by forceps and used to pull the entire IUD through the cervix. But, an IUD may break during the removal process (e.g., an arm may detach from a body of the IUD) resulting in left over fragments of the IUD in the uterus that can require a surgical procedure to remove. Surgery may also be required if the removal strings are drawn back into the uterus such that the strings cannot be grasped for removal. Some couples can sense the removal strings during vaginal intercourse and find the removal strings uncomfortable. Others believe the strings may be linked to a higher risk of infection.

### SUMMARY

The present disclosure describes devices, systems, and methods for female contraception.

In some aspects, an intrauterine device (“IUD”) is provided. The IUD may include a body, an arm, a removal mechanism, and a communication circuit. The body may include a first end and a second end. The arm may extend radially from the first end while the body is inserted in a uterus. The removal mechanism may couple to the second end for removing the intrauterine device from the uterus. The communication circuit may couple to the body or the arm for responding to a first signal by transmitting a second signal.

In some aspects, a method is provided. The method may include transmitting a first signal to a communication circuit coupled to an IUD. The method may further include receiving a second signal from the communication circuit. The method may further include determining the IUD is positioned within a uterus based on the second signal.

In some aspects, an IUD is provided with a body, an arm, a removal mechanism, and a connector. The body may include a first end and a second end. The arm may, in a first position, extend radially from the first end of the body and in a second position be detached from the body. The removal mechanism may couple to the second end for removing the intrauterine device from a uterus. The connector may link the arm to the body such that the arm remains linked to the body in both the first and second position via the connector.

In some aspects, an IUD is provided with a body, an arm, and a removal mechanism. The body may include a first end and a second end. The arm may extend radially from the first end when the body is within a uterus. The removal mechanism may be coupled to the second end and include a magnetic portion for removing the IUD from the uterus.

In some aspects, a method is provided. The method may include inserting a removal tool with a magnetic end through a cervix and into a uterus. The method may further include docking the magnetic end with a magnetic portion of a removal mechanism of an IUD. The method may further include removing the IUD from the uterus with the removal tool.

### BRIEF DESCRIPTION OF THE DRAWINGS

In the following detailed description, embodiments of the disclosure are described referring to the following figures:

FIG. 1 is a cross-sectional diagram of an example of a uterus with an intrauterine device (“IUD”) according to one aspect of the present disclosure.

FIG. 2 is a perspective view of an example of a copper IUD with a communication circuit according to one aspect of the present disclosure.

FIG. 3 is a perspective view of an example of a hormone IUD with a communication circuit according to one aspect of the present disclosure.

FIG. 4 is a block diagram of a communication circuit of an IUD communicatively coupled to an external device via a Near Field Communication (“NFC”) field according to one aspect of the present disclosure.

FIG. 5 is a flow chart of an example of a process for detecting the presence of an IUD, with a communication circuit, within a uterus according to one aspect of the present disclosure.

FIG. 6 is a perspective view of an example of a copper IUD with connectors coupling each arm to the body according to one aspect of the present disclosure.

FIG. 7 is a perspective view of an example of a hormone IUD with connectors coupling each arm to the body according to one aspect of the present disclosure.

FIG. 8 is a cross-sectional diagram of an example of a uterus with a copper IUD with arms extending from the body and with connectors linking each arm to the body according to one aspect of the present disclosure.

FIG. 9 is a cross-sectional diagram of an example of the uterus and IUD in FIG. 8 with arms that have detached from the body, but remain linked to the body via the connectors according to one aspect of the present disclosure.

FIG. 10 is a perspective view of an example of a copper IUD with a removal mechanism with a magnetic portion according to one aspect of the present disclosure.

FIG. 11 is a perspective view of an example of a hormone IUD with a removal mechanism with a magnetic portion according to one aspect of the present disclosure.

FIG. 12 is a side view of a removal tool according to one aspect of the present disclosure.

FIG. 13A is a top view of a removal tool in a closed position according to one aspect of the present disclosure.

FIG. 13B is a side view of a removal tool in a close position according to one aspect of the present disclosure.

FIG. 13C is a top view of a removal tool in an open position according to one aspect of the present disclosure.

FIG. 13D is a side view of a removal tool in an open position according to one aspect of the present disclosure.

FIG. 14 is a flow chart of an example of a process for removing an IUD with a removal mechanism with a magnetic portion according to one aspect of the present disclosure.

FIGS. 15-20 are cross-sectional diagrams of an example of a uterus with a copper IUD being removed using the magnetic portion of a removal mechanism according to one aspect of the present disclosure.

### DETAILED DESCRIPTION

Certain aspects and features relate to an enhanced intrauterine device (“IUD”) that may be more efficient, comfortable, and safe. The IUD may include a communication circuit such that the IUD can be detected wirelessly. The IUD may include connectors that can link a body of the IUD with an arm of the IUD such that the body and arm remain linked if the arm detaches from the body. The IUD may include a removal mechanism with a magnetic portion such that the IUD may dock with a removal tool without the aid of removal strings.

In some aspects, an IUD may include a communication circuit that can transmit a signal for determining the presence of the IUD within a uterus. The communication circuit may be embedded within the IUD or coupled to the surface of the IUD. In some aspects, the communication circuit may be coupled to a power source. In additional or alternative aspects, the communication circuit may be powered by a signal received by the communication circuit. The communication circuit may be communicatively coupled to a memory that may store data regarding the IUD. For example, the data may include a manufacturer of the IUD, a date of production of the IUD, a physician that inserted the IUD, a date the IUD was inserted, and a mode of contraception used by IUD (e.g., copper or hormone). The communication circuit may also be communicatively coupled to one or more sensors that can measure characteristics (e.g., glucose level, body temperature, or iron level) of a user of the IUD. In some examples, the sensors may be on the surface of the IUD such that the sensors are exposed to bodily fluids. In additional or alternative examples, the sensors may be embedded within the IUD. In some aspects, the memory may store data based on the measurements from the sensors.

In response to a first signal, the communication circuit may transmit a second signal indicating the presence of the IUD within the uterus. The first signal may be transmit by a device external to the uterus and the device may determine the IUD is within the uterus by receiving the second signal. The second signal may be modulated to include the data stored in the memory. Additionally or alternatively, the second signal may be modulated to include measurements from the sensors. In some aspects, the device external to the uterus may be a smartphone or another computing device that includes an app for monitoring the IUD. The device may allow a user to display information regarding the IUD as well as periodically remind the user to check the position of the IUD. In additional or alternative aspects, the communication circuit may be a near field communication chip (“NFC”). In additional or alternative aspects, the communication circuit may be communicatively coupled to an antenna for receiving the first signal and transmitting the second signal. In some examples, the coil of copper wire around a copper IUD can be used as the antenna. In additional or alternative examples, an antenna (e.g., copper or silver wire) may be coupled to the IUD. For example, a coil of silver wire may be positioned between the surface of the IUD and the hormone release mechanism of a hormone IUD.

In some aspects, a connector may link an arm of an IUD to a body of the IUD such that a link is maintained if the arm detaches from the body. In some aspects, the arm may be designed to detach from the body as the IUD is removed from the uterus. In additional or alternative aspects, the arm may detach from the body due to a force applied to a joint between the arm and the body. The connector may maintain a link between a detached arm and the body such that the detached arm may be removed with the rest of the IUD.

In some aspects, The connector may be flexible such that the arm can bend (e.g., forming a ‘Y’ shape IUD from a ‘T’ shape IUD) during a removal process. The connector may be a plastic string or any suitable material. In some aspects, the connector may be a portion of the removal strings that attach to an arm of the IUD, pass through the body of the IUD, and extend from the removal mechanism through the cervix. In additional or alternative aspects, the connector may attach to multiple arms and pass through the center of the IUD.

In some aspects, a removal mechanism of the IUD may include a magnetic portion for magnetically docking with a removal tool. In some aspects, the magnetic portion may replace the removal strings. In other aspects, the removal mechanism may include both a magnetic portion and removal strings.

These illustrative examples are given to introduce the reader to the general subject matter discussed here and are not intended to limit the scope of the disclosed concepts. The following sections describe various additional features and examples with reference to the drawings in which like numerals indicate like elements, and directional descriptions are used to describe the illustrative aspects but, like the illustrative aspects, should not be used to limit the present disclosure.

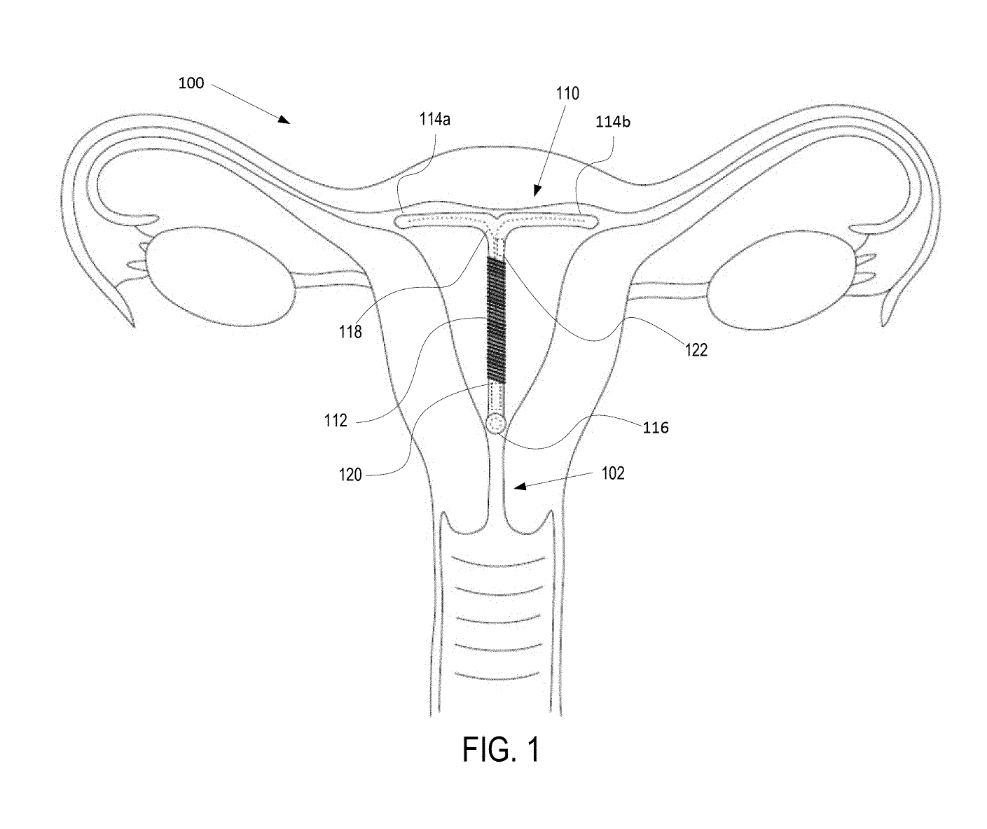

FIG. 1 is a cross-sectional diagram of an example of a uterus 100 with an IUD 110. The IUD 110 includes a body 112 with two arms 114a-b extending from one end of the body 112 to form a ‘T’ shape. On the other end of the body 112 is a removal mechanism 116 with a magnetic portion. The IUD 110 further includes connectors 118 that link the two arms 114a-b to the body 112. The IUD 110 also includes a communication circuit 120 (e.g., a near field communication (“NFC”) chip) and a sensor 122 embedded in the IUD 110. Copper wire 124 coils around the body 112. In some examples, the copper wire 124 can coil around both arms 114a-b.

The IUD 110 may operate as a contraceptive in the uterus 100 by releasing a small amount of copper from the copper wire 124, which can act as a spermicide. In additional or alternative aspects, the IUD 110 may inflame the walls of the uterus 100 and prevent an egg from implanting therein. The copper wire 124 may also be communicatively coupled to the communication circuit 120 to act as an antenna. Although the IUD 110 as illustrated uses copper, other substances may be used (e.g., a hormone). In some examples, a hormone IUD may include an antenna positioned between the surface of the IUD and the hormone release mechanism.

The communication circuit 120 may respond to a first signal transmitted by a device external to the uterus 100 by transmitting a second signal. The second signal may be used by the device to determine that the IUD 110 is within the uterus 100. In some aspects, the second signal may include information about the IUD 110. For example, the second signal may include information related to a manufacturer of the IUD 110, a date of production of the IUD 110, a physician that inserted the IUD 110, a date the IUD 110 was inserted, and the mode of contraception used by IUD 110. In some aspects, information may be stored on a memory communicatively coupled to the communication circuit 120. In additional or alternative aspects, the second signal may include information about a user of the IUD 110. For example, the second signal may include information measured by the sensor 122 such as a glucose level, body temperature, or iron level of the user.

Although the communication circuit 120 and sensor 122 are illustrated as embedded within the IUD 110, a communication circuit and a sensor may be coupled to the surface of an IUD. Furthermore, a communication circuit and a sensor may be coupled to any part of an IUD. In additional or alternative aspects, an IUD may include more than one sensor and some of the sensors may be embedded in the IUD and other sensors can be coupled to the surface of the IUD such that they are exposed to bodily fluids.

The connectors 118 may link arms 114a-b to the body 112 such that if the arms 114a-b detach from the body 112 then the arm 114a-b may still be linked to the body 112 via the connectors 118. For example, an arm 114a-b may detach from the body 112 as the IUD 110 is pulled through a cervix 102 during removal of the IUD 110. The connectors 118 may maintain a link between the arm 114a-b and the body 112 such that the arm 114a-b is removed from the uterus 100 along with the rest of IUD 110. Although the connectors 118 are illustrated as embedded in the IUD 110, in some examples, the connectors 118 are external to the IUD 110 and couple between a surface of the arms 114a-b and a surface of the body 112.

The removal mechanism 116 may include a magnetic portion for docking with a removal tool. The magnetic portion may align a magnetic end of the removal tool with the removal mechanism 116. In some aspects, the magnetic coupling between the magnetic portion and the removal tool may allow the IUD 110 to be pulled through the cervix 102 by pulling out the removal tool. In additional or alternative aspects, the removal mechanism 116 may mechanically lock onto the removal tool after the removal tool has docked with the magnetic portion. As illustrated, the IUD 110 may be entirely housed by the uterus 100. In some aspects, removal strings may extend from the removal mechanism 116 through the cervix 102 and into the vagina.

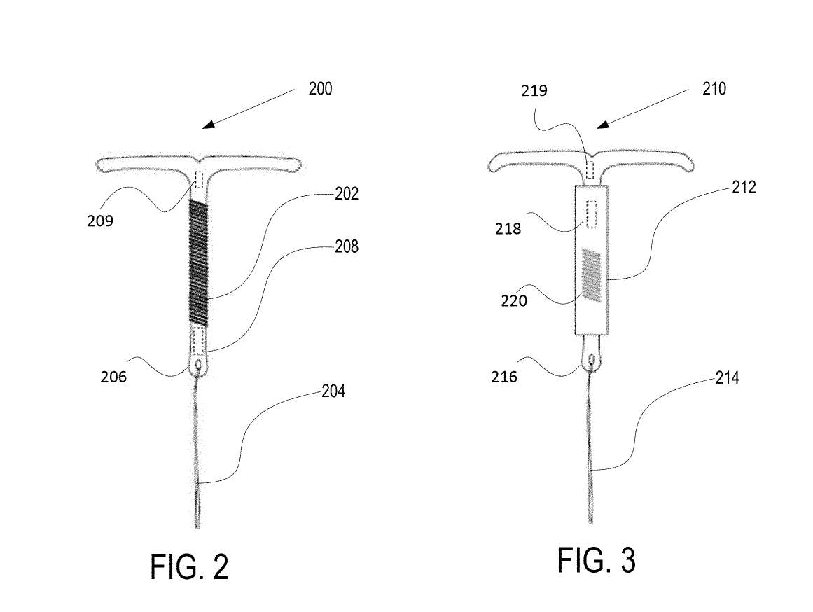

FIGS. 2-3 are perspective views of an example of a copper IUD 200 and hormone IUD 210, each with a communication circuit 208, 218. FIG. 2 illustrates an IUD 200 with copper wire 202, removal string 204, removal mechanism 206, communication circuit 208, and sensor 209. In this example, the copper wire 202 is wrapped around a body of IUD 200. In other examples, the copper wire 202 can be wrapped around a body and two arms of an IUD. The copper wire 202 can gradually release copper that may act as a spermicide while the IUD 200 is within a uterus. FIG. 3 illustrates an IUD 210 with a hormone release 212, removal string 214, removal mechanism 216, communication circuit 218, sensors 219, and an antenna 220. While in the uterus, the hormone release 212 can release a hormone (e.g., levonorgestrel) that may thicken the cervical mucus so that sperm cannot reach an egg.

The removal string 204, 214 may extend from the removal mechanism 206, 216 such that while the IUD 200, 210 is inserted in the uterus, the removal string 204, 214 may extend through the cervix into the vagina. In some aspects, the removal string 204, 214 and removal mechanism 206, 216 may be replaced by a removal mechanism with a magnetic portion. The communication circuit 208, 218 can transmit a signal in response to receiving a signal. In FIG. 2, at least a portion of the copper wire 202 can be communicatively coupled to the communication circuit 208 to act as an antenna. In some examples, as the copper as released into a uterus an impedance of the antenna formed by the copper wire may shift. The shift in the impedance of the copper wire can be analyzed to determine a remaining copper quantity. The IUD 200 can be scheduled to be replaced in response to the remaining copper quantity falling below a threshold value, which can allow for safer and longer use of the IUD 200. In FIG. 3, the antenna 220 may be communicatively coupled to the communication circuit. Although antenna 220 is depicted as positioned between the surface of the IUD 210 and the hormone release 212, an antenna may be positioned outside of a hormone release.

By transmitting a signal from within a uterus, the communication circuit 208, 218 may indicate to a device external to the uterus (e.g., a smartphone) that the IUD 200, 210 is within the uterus. In some aspects, the communication circuit 208, 218 may transmit a modulated signal. The modulated signal may contain data indicating the IUD 200, 210 is within the uterus. In additional or alternative aspects, the data may include information such as a manufacturer of the IUD 200, 210, a date of production of the IUD 200, 210, a physician that inserted the IUD 200, 210, a date of the insertion of the IUD 200, 210, and the mode of contraception used by the IUD 200, 210. The data may also include information regarding the status of the IUD 200, 210 such as an indication that an arm has detached from the body of the IUD 200, 210 or that the removal mechanism 206, 216 is cooperating with a removal tool. In additional or alternative aspects, the data may include measurements from a sensor 209, 219b communicatively coupled to the communication circuit 208, 218. For example, the data may include a glucose level, body temperature, or iron level of a user of the IUD.

The communication circuit 208, 218 and the sensor 209, 219 may be embedded within the IUD 200, 210 or coupled to the surface of the IUD 200, 210. In some aspects, the communication circuit 208, 218 and the sensor 209, 219 may be coupled to a power source. In additional or alternative aspects, the communication circuit 208, 218 and the sensor 209, 219 may be powered by the signal received by the communication circuit 208, 218. The communication circuit 208, 218 may respond to all received signals or only to certain modulated signals. Although FIGS. 2-3 depict IUD 200, 210 with a removal string 204, 214, the presence of the IUD 200, 210 may be detected without the removal string 204, 214.

In some aspects, the communication circuit 208, 218 may be communicatively coupled to a memory that can store the data. The data may be stored to the memory prior to the IUD 200, 210 being inserted into the uterus or may be written to memory while within the uterus. For example, the communication circuit 208, 218 may receive a signal from a device instructing information be stored to the memory. In additional or alternative aspects, a sensor 209, 219 may be communicatively coupled to the memory and may request a measurement be stored to memory. In some aspects, the communication circuit 208 can be a NFC chip that can allow short-range (e.g., a distance of 4 cm) wireless communication.

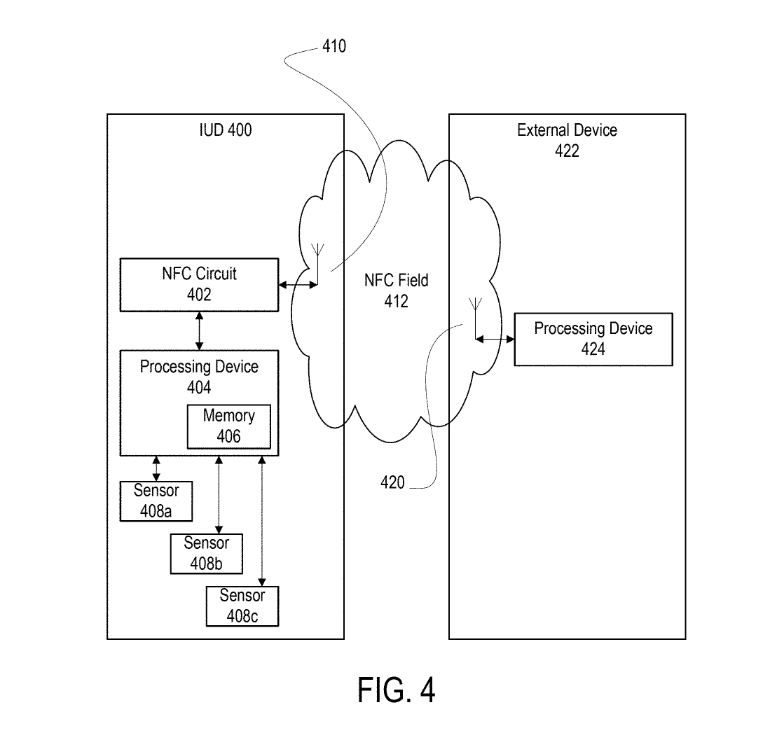

FIG. 4 is a block diagram of an IUD 400 communicatively coupled to an external device 422 via a NFC field 412. The IUD 400 includes an NFC circuit 402, processing device 404 with memory 406, sensors 408a-c, and an antenna 410. The external device (e.g., a smart phone) 422 includes a processing device 424 communicatively coupled to an antenna 420.

The NFC circuit 402 is communicatively coupled to the antenna 410 for receiving signals transmit over the NFC field 412 by the external device 422. The NFC circuit is further communicatively coupled to the processing device 404, which is communicatively coupled to the sensors 408a-c. The signal may be used to power the NFC circuit 402 as well as the processing device 404 and sensors 408a-c. In some aspects, a capacitor may be charged by the signal and used to provide extended power.

The processing device 404 can include any number of processors configured for executing program code stored in the memory 406. Examples of the processing device 404 can include a microprocessor, an application-specific integrated circuit (“ASIC”), a field-programmable gate array (“FPGA”), or other suitable processor. In some aspects, the processing device 404 can be a dedicated processing device used for sending a signal in response to receiving a signal. For example, the processing device 404 can instruct the NFC circuit 402 to transmit a preset response signal. In other aspects, the processing device 404 can perform additional functions such as evaluating measurements from the sensors 408a-c and modulating a response signal to include data related to the IUD 400 and the health of the user of the IUD 400.

The processing device 404 can include (or be communicatively coupled with) a non-transitory computer-readable memory 406. The memory 406 can include one or more memory devices that can store program instructions. The program instructions can include for example, a signal response engine that is executable by the processing device 304 to perform certain operations described herein.

The operations can include determining that a received signal warrants a response. The operations can further include examining measurements from sensors 408a-c and data stored in memory 406 to determine information that should be included in a response signal. The operations can further include instructing the NFC circuit 402 to transmit a response signal that includes the information.

The memory 406 can also store information about the IUD 400 such as a manufacturer of the IUD 400, a date of production of the IUD 400, a physician that inserted the IUD 400, a date the IUD 400 was inserted, and the mode of contraception used by IUD 400. In additional or alternative aspects, the memory 406 can store measurements taken by the sensors 408a-c.

In some examples, the sensors 408a-c can be embedded within the IUD 400 and on the surface of the IUD 400 such that the sensors 408a-c are exposed to bodily fluids. The sensors 408a-c embedded in the IUD 400 can measure characteristics of the user of the IUD 400 such as body temperature. The sensors 408a-c exposed to bodily fluids can measure characteristics of the user of the IUD 400 such as glucose level and iron level.

The processing device 424 in the external device 422 can receive a signal transmit by the IUD 400 via the antenna 420. The processing device 424 can use the received signal to determine the presence of the IUD 400. In some aspects, the processing device 424 can further use the signal to determine characteristics of the IUD 400 and the health of the user of the IUD 400. In additional or alternative aspects, the processing device 424 may instruct the external device 422 to display an analysis of the information determined from the received signal. In additional or alter aspects, the processing device 424 may instruct the external device 422 to communicate with another device or an emergency medical service to provide assistance to the user of the IUD 400. Although FIG. 4 illustrates the IUD 400 with an NFC circuit 402, any wireless communication circuit may be used to communicate over a wireless network with an external device.

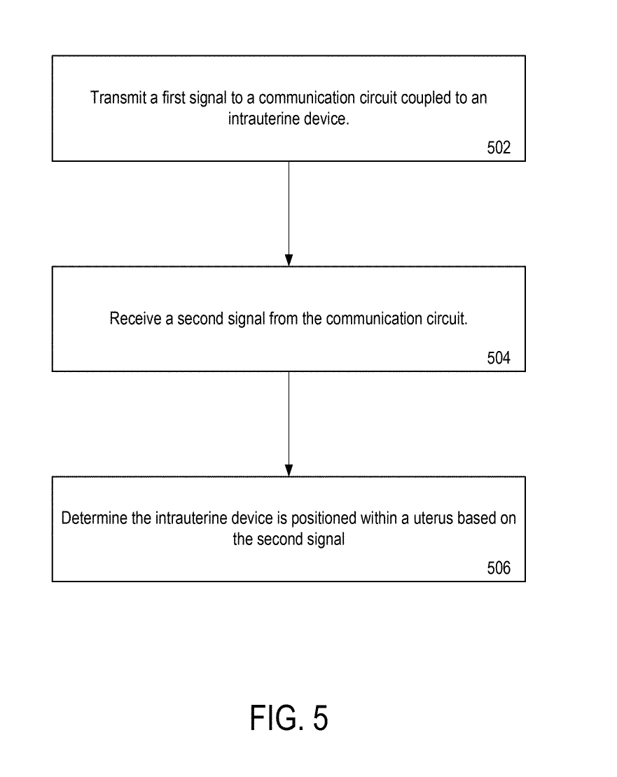

FIG. 5 is a flow chart of an example of a process for detecting the presence of an IUD, including a communication circuit, within a uterus.

In block 502, a first signal is transmit to a communication circuit coupled to an IUD. The first signal may be a wireless signal transmitted by a device (e.g., a smartphone) separate from the IUD. In some aspects, the first signal may be received by an antenna. In some examples, the antenna may be copper wire for releasing copper by a copper IUD. In additional or alternative examples, the antenna may be positioned between a surface of a hormone IUD and a hormone release. In some aspects, the first signal powers the communication circuit and causes the communication circuit to transmit a second signal in response. In additional or alternative aspects, the first signal may include instructions to transmit a second signal.

In block 504, a second signal is received from the communication circuit. The second signal may be received by the device that transmitted the first signal or a separate device. In some aspects, the second signal is a short pulse. In additional or alternative aspects, the second signal may be a modulated to include data that indicates the IUD is within the uterus.

The second signal may also be modulated to include data about the IUD or the user of the IUD. For example, the second signal may be modulated to include data on a manufacturer of the IUD, a date of production of the IUD, a physician that inserted the IUD, a date the IUD was inserted, and a mode of contraception used by the IUD. In additional or alternative examples, the second signal may include data about the current status of the IUD. For example, the data may indicate an arm has detached from a body of the IUD or a removal mechanism of the IUD is cooperating with a removal tool. In additional or alternative aspects, the second signal may include data about the condition of the user of the IUD, such as the glucose level, body temperature, or iron level of the user.

In block 506, the IUD may be determined to be positioned within the uterus based on the second signal. In some aspects, the device that received the second signal may be positioned such that receiving the signal allows the determination that the IUD is within the uterus. In additional or alternative aspects, a processing device may determine from data included in the second signal that the IUD is within the uterus.

The processing device may determine additional information from the data included in the second signal. In some aspects, the processing device may determine medical action should be taken by the user and may display an analysis of the data to the user. For example, the processing device may determine a diabetic user has low blood sugar based on measurements transmitted as part of the data. The processing device may display the user's blood sugar level as well as a recommended insulin dosage. In additional or alternative aspects, the processing device may communicate to an additional device (e.g., an insulin pump) and instruct the additional device to perform an action (e.g., inject the user with insulin). In additional or alternative aspects, the processing device may determine that emergency medical attention should be taken and may contact a hospital or emergency medical service provider.

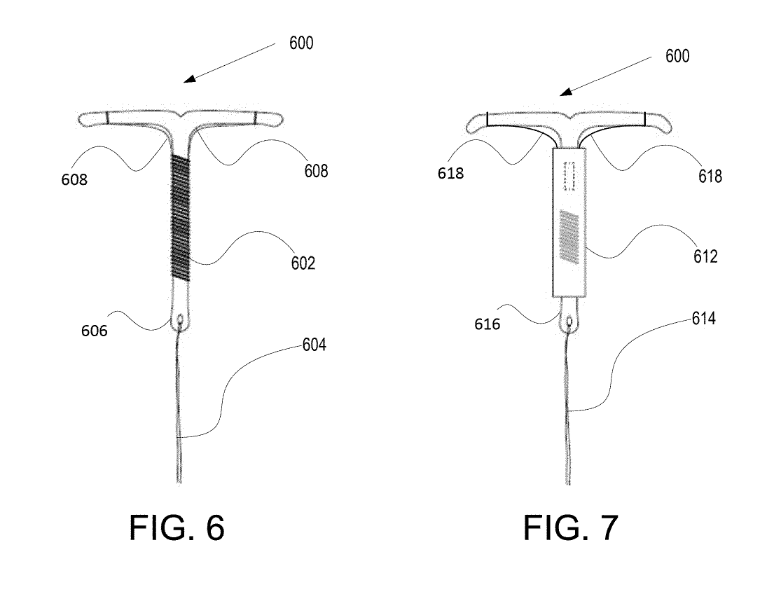

FIGS. 6-7 are perspective views of an example of a copper IUD 600 and a hormone IUD 610 with connectors 608, 618 linking each arm to a respective body. Similar to the IUD 200 in FIG. 2, IUD 600 includes copper wire 602, a removal string 604, and a removal mechanism 606. Similar to the IUD 210 in FIG. 3, IUD 610 includes a hormone release 612, removal string 614, and removal mechanism 616. The IUD 600, 610 also includes connectors 608, 618 that link the each arm to the body.

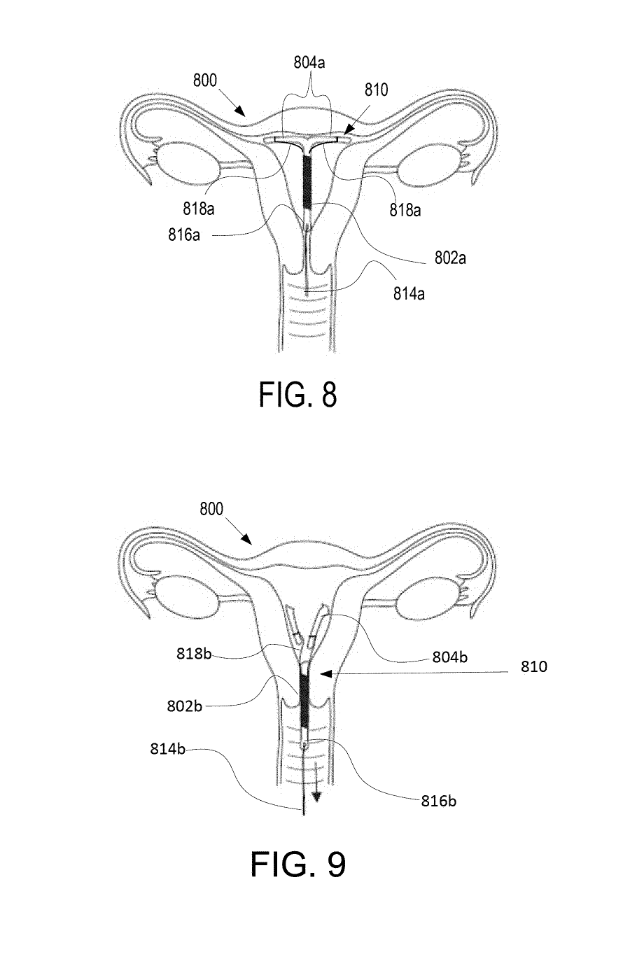

In some aspects, an arm may extend radially from a body of the IUD 600, 610 in a first position. And, the arm may be detached from the body in a second position. The connectors 608, 618 can maintain a link between the arm and the body in both the first and second position. FIGS. 8-9 depict the arms in a first and second position.

FIG. 8 is a cross-sectional diagram of an example of a uterus 800 with an IUD 810 with arms 804a in the first position (e.g. extending from the body 802a) and with connectors 818a coupling each arm 804a to the body 802a. FIG. 9 is a cross-sectional diagram of an example of the uterus 800 in FIG. 8 with the IUD 810 with arms 804b in the second position (e.g., detached from the body 802b) but linked to the body 802b via the connectors 818b. In some aspects, the arms 804a may be in the first position while inserted in the uterus 800 as illustrated in FIG. 8 with the removal mechanism 816a entirely within the uterus and the removal string 814a extending from the removal mechanism 816a through the cervix and into the vagina. The arms 804a may move to a second position during removal of the IUD 810 from the uterus 800. FIG. 9 illustrates a portion of the removal process during which the removal mechanism 816b has been pulled into the vagina via the removal string 814b. The connectors 818b may cause the arms 804b to be removed with the IUD 810. Although FIGS. 8-9 depict a copper IUD 810, any suitable IUD may be used with the connectors 818a-b.

Returning to FIGS. 6-7, although the connectors 608, 618 are depicted as individual links between the arms and the body. A single connector may couple to one arm, pass through a hole in the body of the IUD, and couple to a second arm. In this example, the hole may be smaller than an arm and may link the arms to the body such that the arms can be removed with the IUD while the arms are detached from the body. In additional or alternative aspects, the IUD 600, 610 may have one arm or more than two arms and connectors may link one or more of the arms to the body.

In additional or alternative aspects, the arms of the IUD 600, 610 may be flexible such that the arms can bend to form a ‘Y’ shaped IUD. The connectors 608, 618 may be a flexible material or possess sufficient slack to allow the arms to bend. In some aspects, the connector 608, 618 may be an extension of the removal string 604, 614. For example, the removal string 604, 614 may attach to an arm, couple to the removal mechanism 606, 616 and be configured to extend through a cervix while the IUD 600, 610 is inserted in a uterus. In additional or alternative aspects, the removal mechanism 606, 616 and removal string 604, 614 may be replaced by a removal mechanism with a magnetic portion.

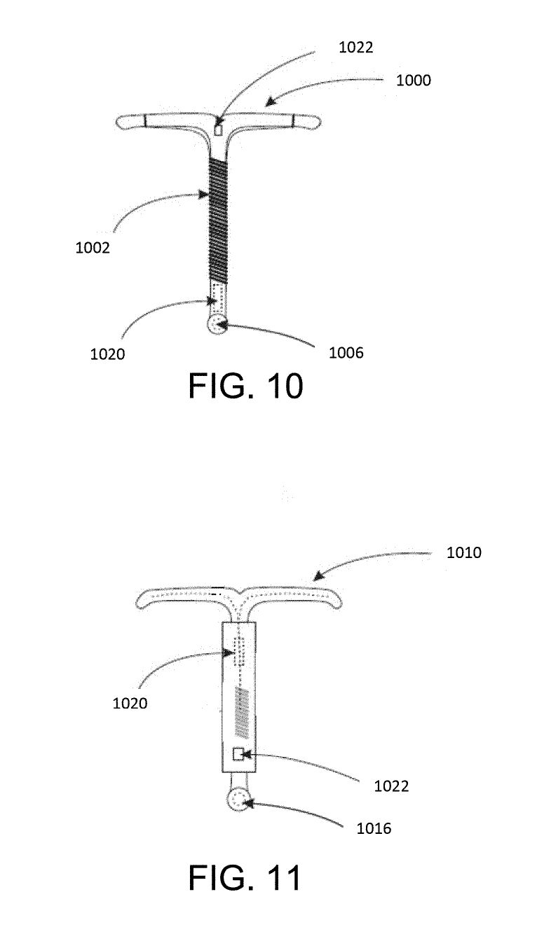

FIGS. 10-11 are perspective view of an example of a copper IUD 1000 and hormone IUD 1010 with a removal mechanism 1006, 1016 with a magnetic portion. IUD 1000 includes copper wiring 1002 wrapped around a body of the IUD 1000 and two arms of the IUD 1000. The IUD 1010 includes a hormone release 1012 coupled to a body of the IUD 1010. The magnetic portion of the removal mechanism 1006, 1016 may be generated using an electromagnet or may have naturally magnetic properties. The copper IUD 1000 and hormone IUD 1010 can have a communication circuit 1020 and a sensor 1022, which can include circuitry or insulation to prevent the magnetic portion of the removal mechanism 1006, 1016 from interfering with communications or measurements respectively.

In some aspects, the magnetic portion of the removal mechanism 1006, 1016 may have a strong enough magnetic force that the removal mechanism 1006, 1016 can couple to a magnetic end of a removal tool. By coupling with the removal tool, the IUD 1000, 1010 can be removed from a uterus as the removal tool is pulled out of the uterus. In additional or alternative aspects, an additional locking mechanism may be used to couple the removal tool to the IUD 1000, 1010.

For example, the removal mechanism 1006, 1016 may include a first portion and a second portion. The second portion may be farther axially from the body of the intrauterine device 1000, 1010 than the first portion and may extend farther radially (e.g., have a greater circumference) than the first portion. The removal tool may include a loop that can tighten around the first portion while the removal tool is docked with the removal mechanism 1006, 1016. Once tightened the loop may have a smaller diameter than the second portion such that the removal tool is coupled to the IUD 1000, 1010. Although removal mechanism 1006, 1016 is illustrated as having a spherical shape, and suitable shape may be used. In some aspects, the removal mechanism 1006, 1016 can reduce the risk of infection, reduce interference with sexual activities, and improve privacy by reducing the visibility to non-users than an IUD is being used.

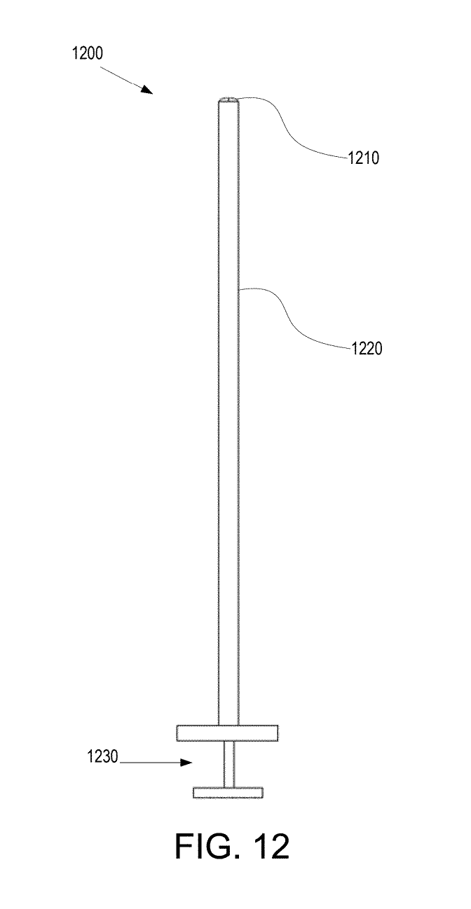

FIG. 12 is a side view of an example of a removal tool 1200 for removing an IUD. In this example, the removal tool 1200 includes a body 1220 with a grasping mechanism 1210 on one end and a control mechanism 1230 on another end. The body 1220 can be long enough to extend through a woman's cervix and into her uterus. The grasping mechanism 1210 can move between an open state and a closed state. In the open state, the grasping mechanism 1210 can create an opening. In some examples, the grasping mechanism 1210 can include one or more members that can fold radially outward to form the opening. In the closed position, the grasping mechanism can press radially inward closing the opening or grasping onto a removal mechanism of an IUD positioned in the opening. The control mechanism 1230 can be coupled to the grasping mechanism 1210 such that the control mechanism 1230 can control the state of the grasping mechanism 1210. The control mechanism 1230 includes a handle that can move between an extended position and a depressed position. In the extended position, the control mechanism 1230 causes the grasping mechanism 1210 to close. In the depressed position, the control mechanism 1230 causes the grasping mechanism 1210 to open.

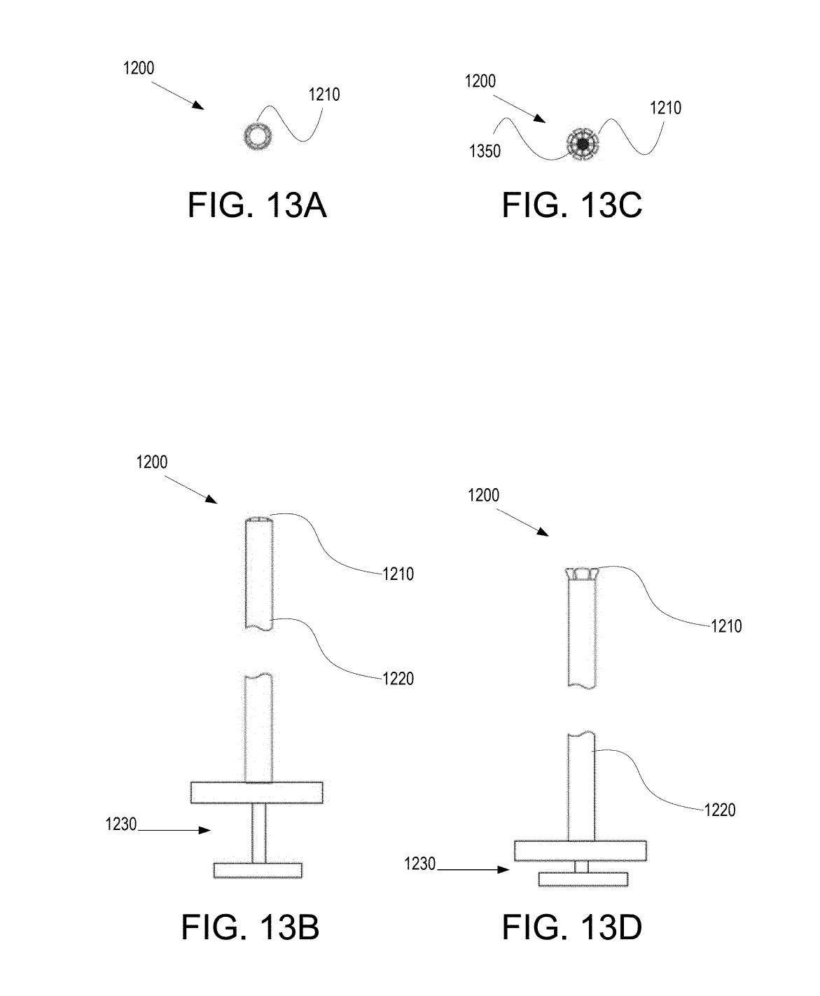

FIGS. 13A-D depict an example of the removal tool 1200 in a closed position and an open position. FIG. 13A depicts a top view of the grasping mechanism 1210 in a closed position and FIG. 13C depicts a top view of the grasping mechanism 1210 in an open position. A magnet 1350 is illustrated in FIG. 13C and can have an opposite polarity of a magnetic removal mechanism in an IUD. The magnet 1350 can be used to locate and position the IUD within the opening formed by the grasping mechanism 1210. In some examples, the magnet 1350 can be an electromagnet that receives power in response to the grasping mechanism 1210 moving to an open state. FIG. 13B depicts a side view of a portion of the removal tool 1200 in a closed position and FIG. 13 D depicts a side view of a portion of the removal tool 1200 in an open position.

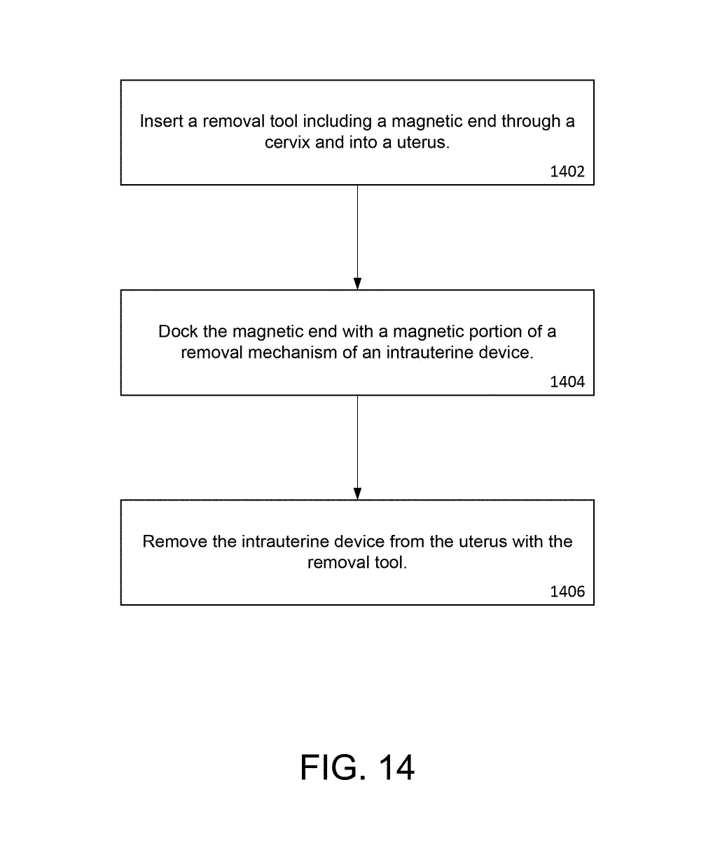

FIG. 14 is a flow chart of an example of a process for removing an IUD with a removal mechanism with a magnetic portion.

In block 1402, a removal tool including a magnetic end is inserted through a cervix and into a uterus. In some aspects, the removal tool may include an electromagnet such that when power is applied removal tool, a magnetic force is generated at the magnetic end.

In block 1404, the magnetic end of the removal tool docks with the magnetic portion of the removal mechanism of the IUD. In some aspects, the magnetic force between the removal mechanism and the magnetic end may be strong enough to couple the removal tool with the removal mechanism. In additional or alternative aspects, the magnetic force may allow the removal tool to dock with the removal mechanism such that the removal tool can couple to the removal mechanism using a mechanical locking mechanism. For example, the removal tool may include a loop on the magnetic end that may tighten around the removal mechanism once the removal tool has docked with the removal mechanism.

In block 1406, the IUD is removed from the uterus with the removal tool. By pulling the removal tool, while docked with the removal mechanism, through the cervix the IUD is pulled through the cervix.

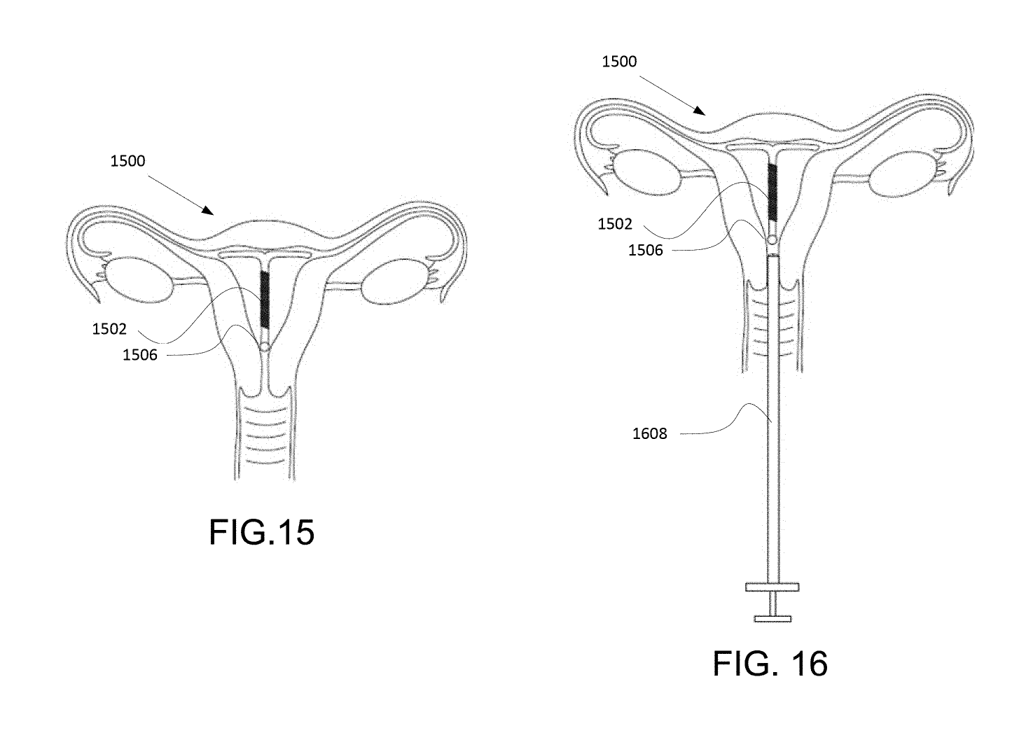

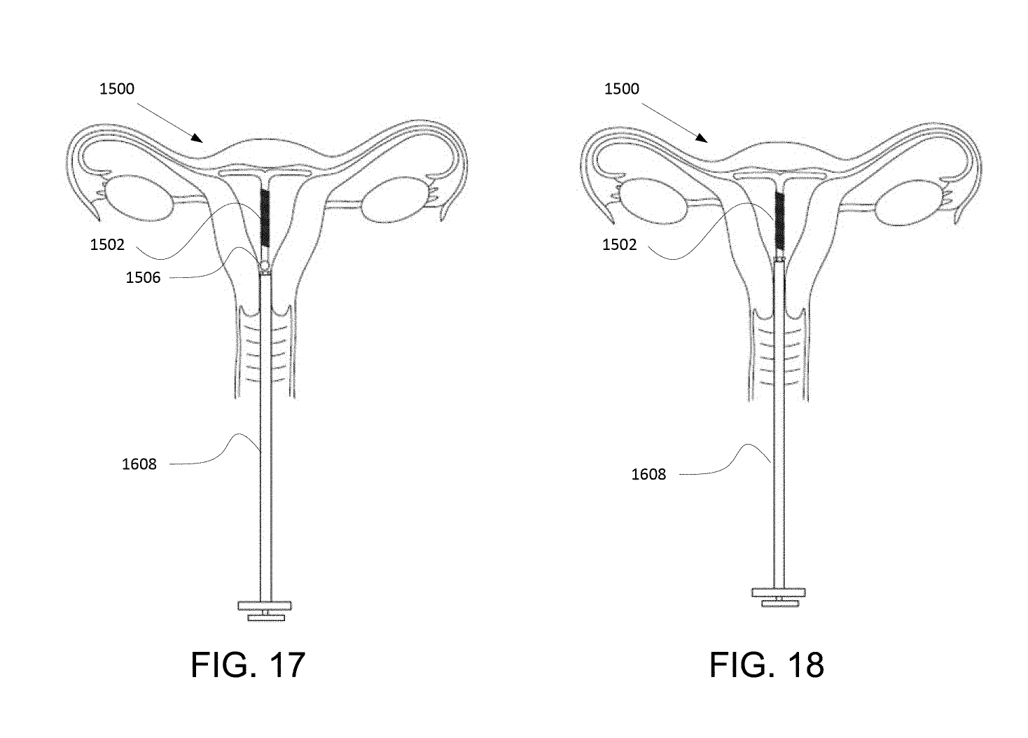

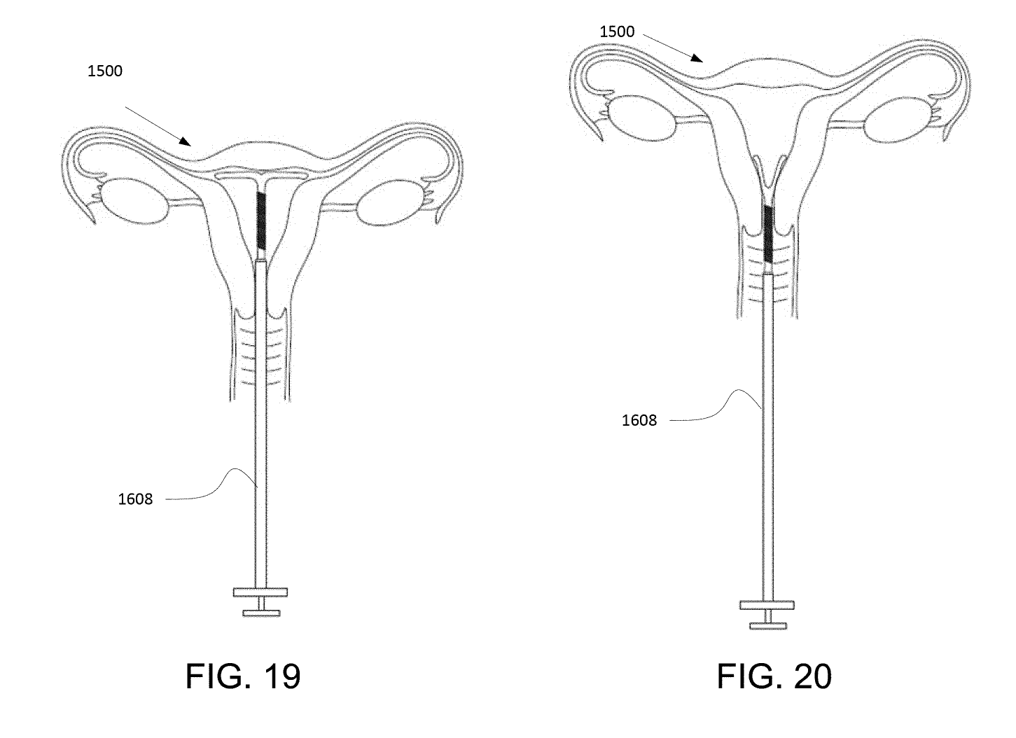

FIGS. 15-20 are cross-sectional diagrams of an example of a uterus 1500 during removal of a copper IUD 1502 using a removal tool 1608 docked to the magnetic portion of a removal mechanism 1506. As illustrated, the removal mechanism 1506 can be coupled to one end of a body of the IUD 1502. The removal mechanism 1506 includes a magnetic portion that can dock with a magnetic end of the removal tool 1608. As the removal tool 1608 is pulled out of the uterus 1500 and through the cervix, the IUD 1502 is pulled down through the uterus 1500.

As the IUD 1502 is removed from the uterus 1500, the arms of the IUD 1502 may bend giving the IUD 1502 a ‘Y’ shape. In some aspects, connectors may link the arms to the body of the IUD 1502 such that the arms and body remain linked if the arms detach from the body. In additional or alternative aspects, the IUD 1502 may include a communication circuit that can transmit a wireless signal from inside the uterus 1500 to a device external to the uterus 1500. The signal may include an indication when the removal tool 1608 is docked with the IUD 1502.

In some aspects, an enhanced intrauterine device is provided according to one or more of the following examples:

### Example #1

An intrauterine device include a body, at least one arm, a removal mechanism, and a communication circuit. The body can include a first end and a second end. The at least one arm can extend radially from the first end while the body is inserted in a uterus. The removal mechanism can be coupled to the second end for removing the intrauterine device from the uterus. The communication circuit can be coupled to at least one of the body and the at least one arm for responding to a first signal by transmitting a second signal.

### Example #2

The intrauterine device of Example #1, can further include one or more sensors communicatively coupled to the communication circuit for measuring health data of a user of the intrauterine device.

### Example #3

The intrauterine device of Example #2, can further feature a sensor of the one or more sensors being embedded within the intrauterine device for measuring a body temperature of the user.

### Example #4

The intrauterine device of Example #2, can further feature a sensor of the one or more sensors being exposed to bodily fluids for measuring at least one of a glucose level of the user and an iron level of the user.

### Example #5

The intrauterine device of Example #2, can further feature a sensor of the one or more sensors being powered by the first signal.

### Example #6

The intrauterine device of Example #1, can further include further memory communicatively coupled to the communication circuit for storing data. The second signal can include at least a portion of the data.

### Example #7

The intrauterine device of Example #6, can further feature the portion of the data including at least one of a manufacturer of the intrauterine device, a date of production of the intrauterine device, a physician that performed an insertion of the intrauterine device, a date of insertion of the intrauterine device, and a type of the intrauterine device.

### Example #8

The intrauterine device of Example #1, can further feature the communication circuit being a near field communication chip and being powered by the first signal.

### Example #9

The intrauterine device of Example #1, can further include a copper component coupled to at least one of the body and the at least one arm for releasing copper into the uterus.

### Example #10

The intrauterine device of Example #9, can further feature the copper component being further for operating as an antenna for receiving the first signal and for transmitting the second signal.

### Example #11

The intrauterine device of Example #1, can further include a hormone component coupled to at least one of the body and the at least one arm for releasing a hormone into the uterus.

### Example #12

The intrauterine device of Example #11, can further include an antenna coupled to at least one of the body and the at least one arm for receiving the first signal and for transmitting the second signal.

### Example #13

The intrauterine device of Example #1, can further feature the at least one arm in a first position being directly coupled to the body and in a second position being detached from the body. The intrauterine device can further include at least one connector for linking the at least one arm to the body such that the at least one arm remains linked to the body in both the first position and the second position via the at least one connector.

### Example #14

The intrauterine device of Example #13, can further feature the communication circuit being further for modulating the second signal to indicate the at least one arm is in the second position.

### Example #15

The intrauterine device of Example #1, can further feature the removal mechanism including a magnetic portion for magnetically docking to a removal tool.

### Example #16

The intrauterine device of Example #15, can further feature the communication circuit being for modulating the second signal to indicate the magnetic portion is docked to the removal tool.

### Example #17

A method can include transmitting a first signal to a communication circuit coupled to an intrauterine device. The method can further include receiving a second signal from the communication circuit. The method can further include determining the intrauterine device is positioned within a uterus based on the second signal.

### Example #18

The method of Example #17, can further feature the communication circuit being communicatively coupled to a memory that stores data and the second signal includes at least a portion of the data.

### Example #19

The method of Example #18, can further include determining at least one of a manufacturer of the intrauterine device, a date of production of the intrauterine device, a physician that performed an insertion of the intrauterine device, a date of insertion of the intrauterine device, and a type of the intrauterine device from the second signal.

### Example #20

The method of Example #17, can further feature the communication circuit being communicatively coupled to one or more sensors that measure health information associated with a user of the intrauterine device and the second signal includes at least a portion of the health information.

### Example #21

The method of Example #17, can further feature the communication circuit being part of a near field communication chip.

### Example #22

The method of Example #17, can further feature the intrauterine device including a copper component for releasing copper into the uterus.

### Example #23

The method of Example #22, can further feature transmitting the first signal to the communication circuit further including transmitting the first signal to the copper component that operates as an antenna and is communicatively coupled to the communication circuit.

### Example #24

The method of Example #17, can further feature the intrauterine device including a hormone component for releasing hormones into the uterus.

### Example #25

The method of Example #17, can further feature the intrauterine device including at least one arm that in a first position extends from a body of the intrauterine device and in a second position is detached from the body, and at least one connector for linking the at least one arm to the body such that the at least one arm remains linked to the body in both the first position and the second position via the at least one connector.

### Example #26

The method of Example #25, can further include determining the at least one arm is in the second position based on the second signal.

### Example #27

The method of Example #17, can further feature the intrauterine device including a removal mechanism with a magnetic portion for docking with a removal tool for removing the intrauterine device from the uterus.

### Example #28

The method of Example #27, can further include determining that the magnetic portion is docked with the removal tool based on the second signal.

### Example #29

The method of Example #17, can further include displaying information based on the second signal for making medical decisions.

### Example #30

The method of Example #17, can further include communicating information based on the second signal to another device to request a user of the intrauterine device receive medical assistance.

### Example #31

An intrauterine device can include a body, at least one arm, a removal mechanism, and at least one connector. The body can include a first end and a second end. The at least one arm can, in a first position, extend radially from the first end and, in a second position, be detached from the body. The removal mechanism can be coupled to the second end for removing the intrauterine device from a uterus. The at least one connector can link the at least one arm to the body such that the at least one arm remains linked to the body in both the first position and the second position via the at least one connector.

### Example #32

The intrauterine device of Example #31, can further feature the at least one arm in the first position being configured to move to the second position in response to removing the intrauterine device from the uterus.

### Example #33

The intrauterine device of Example #31, can further include a communication circuit coupled to at least one of the body and the at least one arm for responding to a first signal by transmitting a second signal.

### Example #34

The intrauterine device of Example #33, can further include a memory communicatively coupled to the communication circuit for storing data. The second signal can include at least a portion of the data.

### Example #35

The intrauterine device of Example #34, can further feature the portion of the data including at least one of a manufacturer of the intrauterine device, a date of production of the intrauterine device, a physician that performed an insertion of the intrauterine device, a date of insertion of the intrauterine device, and a type of the intrauterine device.

### Example #36

The intrauterine device of Example #33, can further include one or more sensors communicatively coupled to the communication circuit for measuring health data associated with a user of the intrauterine device. The second signal can include at least a portion of the health data.

### Example #37

The intrauterine device of Example #36, can further feature a sensor of the one or more sensors being exposed to bodily fluids for measuring at least one of glucose level of the user and iron level of the user.

### Example #38

The intrauterine device of Example #36, can further feature the communication circuit being a near field communication chip and at least one of the communication circuit and at least one sensor of the one or more sensors being powered by the first signal.

### Example #39

The intrauterine device of Example #33, can further feature the communication circuit being further for modulating the second signal to indicate the at least one arm is in the second position.

### Example #40

The intrauterine device of Example #33, can further include a copper component coupled to at least one of the body and the at least one arm for releasing copper into the uterus, and communicatively coupled to the communication circuit for operating as an antenna.

### Example #41

The intrauterine device of Example #31, can further feature the removal mechanism including a magnetic portion for magnetically docking with a removal tool.

### Example #42

The intrauterine device of Example #31, can further include a copper component coupled to at least one of the body and the at least one arm for releasing copper into the uterus.

### Example #43

The intrauterine device of Example #31, can further include a hormone component coupled to at least one of the body and the at least one arm for releasing a hormone into the uterus.

### Example #44

An intrauterine device including a body, at least one arm, and a removal mechanism. The body including a first end and a second end. The at least one arm extending radially from the first end when the body is within a uterus. The removal mechanism having a magnetic portion coupled to the second end for removing the intrauterine device from the uterus.

### Example #45

The intrauterine device of Example #44, can further feature the removal mechanism being further for magnetically docking with a removal tool.

### Example #46

The intrauterine device of Example #45, can further feature the removal mechanism including a first portion and a second portion and the second portion being farther from the body than the first portion and the second portion having a greater circumference than the first portion for locking to the removal tool when a loop attached to the removal tool is tightened around the first portion such that the loop has a smaller diameter than the second portion.

### Example #47

The intrauterine device of Example #44, can further include a communication circuit coupled to at least one of the body and the at least one arm for responding to a first signal by transmitting a second signal.

### Example #48

The intrauterine device of Example #47, can further include memory communicatively coupled to the communication circuit for storing data. The second signal can include at least a portion of the data.

### Example #49

The intrauterine device of Example #48, can further feature the portion of the data including at least one of a manufacturer of the intrauterine device, a date of production of the intrauterine device, a physician that performed an insertion of the intrauterine device, a date of insertion of the intrauterine device, and a type of the intrauterine device.

### Example #50

The intrauterine device of Example #47, can further include one or more sensors communicatively coupled to the communication circuit for measuring health data associated with a user of the intrauterine device. The second signal can include at least a portion of the health data.

### Example #51

The intrauterine device of Example #50, can further feature a sensor of the one or more sensors being exposed to bodily fluids for measuring at least one of glucose level of the user and iron level of the user.

### Example #52

The intrauterine device of Example #50, can further feature the communication circuit being a near field communication chip and at least one of the communication circuit and at least one sensor of the one or more sensors being powered by the first signal.

### Example #53

The intrauterine device of Example #47, can further feature the communication circuit being further for modulating the second signal to indicate the magnetic portion is docked with a removal tool.

### Example #54

The intrauterine device of Example #44, can further feature the at least one arm in a first position being directly coupled to the body and in a second position being detached from the body. The intrauterine device can further include at least one connector for linking the at least one arm to the body such that the at least one arm remains linked to the body in both the first position and the second position via the at least one connector.

### Example #55

The intrauterine device of Example #44, can further include a copper component coupled to at least one of the body and the at least one arm for releasing copper into the uterus.

### Example #56

The intrauterine device of Example #44, can further include a hormone component coupled to at least one of the body and the at least one arm for releasing a hormone into the uterus.

### Example #57

A method can include inserting a removal tool including a magnetic end through a cervix and into a uterus. The method can further include docking the magnetic end with a magnetic portion of a removal mechanism of an intrauterine device within the uterus. The method can further include removing the intrauterine device from the uterus with the removal tool.

### Example #58

The method of Example #57, can further feature the removal mechanism including a first portion and a second portion and the second portion being farther from a body of the intrauterine device than the first portion and having a greater circumference than the first portion. Docking can include tightening a loop attached to the removal tool around the first portion such that the loop has a smaller diameter than the second portion.

### Example #59

The method of Example #57, can further feature docking the removal tool further including activating an indicator light for indicating the removal tool has coupled with the magnetic portion.

### Example #60

The method of Example #57, can further feature the intrauterine device further including a communication circuit for responding to a first signal by transmitting a second signal.

### Example #61

The method of Example #60, can further feature the second signal being modulated to indicate that the removal tool is docked to the removal mechanism.

### Example #62

The method of Example #60, can further feature the communication circuit being communicatively coupled to one or more sensors that measure health information associated with a user of the intrauterine device and the second signal includes at least a portion of the health information.

### Example #63

The method of Example #60, can further feature transmitting the first signal to the communication circuit further including transmitting the first signal to a copper component that operates as an antenna and is communicatively coupled to the communication circuit.

### Example #64

The method of Example #57, can further feature the intrauterine device including at least one arm that in a first position extends from a body of the intrauterine device and in a second position is detached from the body. The intrauterine device can further include at least one connector for linking the at least one arm to the body such that the at least one arm remains linked to the body in both the first position and the second position via the at least one connector.

### Example #65

The method of Example #57, can further feature the intrauterine device including a copper component for releasing copper into the uterus.

### Example #66

The method of Example #57, can feature the intrauterine device including a hormone component for releasing hormones into the uterus.

The foregoing description of certain examples, including illustrated examples, has been presented only for the purpose of illustration and description and is not intended to be exhaustive or to limit the disclosure to the precise forms disclosed. Numerous modifications, adaptations, and uses thereof will be apparent to those skilled in the art without departing from the scope of the disclosure.

What is claimed is:

1. An intrauterine device comprising:
a body including a first end and a second end;
at least one arm for extending radially from the first end while the body is inserted in a uterus;
a removal mechanism coupled to the second end for removing the intrauterine device from the uterus; and
a communication circuit coupled to at least one of the body and the at least one arm for responding to a first signal by transmitting a second signal.

2. The intrauterine device of claim 1, further comprising:
one or more sensors communicatively coupled to the communication circuit for measuring health data of a user of the intrauterine device, wherein a first sensor of the one or more sensors is embedded within the intrauterine device for measuring a body temperature of the user, wherein a second sensor of the one or more sensors is exposed to bodily fluids for measuring at least one of a glucose level of the user and an iron level of the user, wherein a third sensor of the one or more sensors is powerable by the first signal.

3. The intrauterine device of claim 1, further comprising:
memory communicatively coupled to the communication circuit for storing data, and wherein the second signal includes at least a portion of the data, wherein the portion of the data comprises at least one of a manufacturer of the intrauterine device, a date of production of the intrauterine device, a physician that performed an insertion of the intrauterine device, a date of insertion of the intrauterine device, and a type of the intrauterine device.

4. The intrauterine device of claim 1, wherein the communication circuit is a near field communication chip and is powerable by the first signal.

5. The intrauterine device of claim 1, further comprising:
a copper component coupled to at least one of the body and the at least one arm for releasing copper into the uterus, wherein the copper component is further for operating as an antenna for receiving the first signal and for transmitting the second signal.

6. The intrauterine device of claim 1, further comprising:
a hormone component coupled to at least one of the body and the at least one arm for releasing a hormone into the uterus; and
an antenna coupled to at least one of the body and the at least one arm for receiving the first signal and for transmitting the second signal.

7. The intrauterine device of claim 1, wherein the at least one arm in a first position is directly coupled to the body and in a second position is detached from the body, and the intrauterine device further comprises at least one connector for linking the at least one arm to the body such that the at least one arm remains linked to the body in both the first position and the second position via the at least one connector, wherein the communication circuit is further for modulating the second signal to indicate the at least one arm is in the second position, wherein the removal mechanism includes a magnetic portion for magnetically docking to a removal tool, wherein the communication circuit is further for modulating the second signal to indicate the magnetic portion is docked to the removal tool.

8. A method comprising:
transmitting a first signal to a communication circuit coupled to an intrauterine device;
receiving a second signal from the communication circuit; and
determining the intrauterine device is positioned within a uterus based on the second signal.

9. The method of claim 8, further comprising:
determining at least one of a manufacturer of the intrauterine device, a date of production of the intrauterine device, a physician that performed an insertion of the intrauterine device, a date of insertion of the intrauterine device, and a type of the intrauterine device from the second signal.

10. The method of claim 8, wherein the intrauterine device comprises a copper component for releasing copper into the uterus, wherein transmitting the first signal to the communication circuit further comprises transmitting the first signal to the copper component that operates as an antenna and is communicatively coupled to the communication circuit.

11. The method of claim 8, wherein the intrauterine device comprises at least one arm that in a first position extends from a body of the intrauterine device and in a second position is detached from the body, and at least one connector for linking the at least one arm to the body such that the at least one arm remains linked to the body in both the first position and the second position via the at least one connector, the method further comprising determining the at least one arm is in the second position based on the second signal.

12. The method of claim 8, wherein the intrauterine device comprises a removal mechanism with a magnetic portion for docking with a removal tool for removing the intrauterine device from the uterus, the method further comprising determining that the magnetic portion is docked with the removal tool based on the second signal.

13. The method of claim 8, further comprising:
displaying information based on the second signal for making medical decisions; and
communicating information based on the second signal to another device to request a user of the intrauterine device receive medical assistance.

14. An intrauterine device comprising:
a body including a first end and a second end;
at least one arm that in a first position extends radially from the first end and in a second position is detached from the body;
a removal mechanism coupled to the second end for removing the intrauterine device from a uterus; and
at least one connector for linking the at least one arm to the body such that the at least one arm remains linked to the body in both the first position and the second position via the at least one connector.

15. The intrauterine device of claim 14, wherein the at least one arm in the first position is configured to move to the second position in response to removing the intrauterine device from the uterus.

16. The intrauterine device of claim 14, further comprising:
a communication circuit coupled to at least one of the body and the at least one arm for responding to a first signal by transmitting a second signal;
memory communicatively coupled to the communication circuit for storing data, and wherein the second signal includes at least a portion of the data, wherein the portion of the data comprises at least one of a manufacturer of the intrauterine device, a date of production of the intrauterine device, a physician that performed an insertion of the intrauterine device, a date of insertion of the intrauterine device, and a type of the intrauterine device; and
one or more sensors communicatively coupled to the communication circuit for measuring health data associated with a user of the intrauterine device, and wherein the second signal includes at least a portion of the health data, wherein a sensor of the one or more sensors is exposable to bodily fluids for measuring at least one of glucose level of the user and iron level of the user, wherein the communication circuit is a near field communication chip and at least one of the communication circuit and at least one sensor of the one or more sensors are powerable by the first signal,
wherein the removal mechanism includes a magnetic portion for magnetically docking with a removal tool.

17. The intrauterine device of claim 16, wherein the communication circuit is further for modulating the second signal to indicate the at least one arm is in the second position, the intrauterine device further comprising a copper component coupled to at least one of the body and the at least one arm for releasing copper into the uterus, and communicatively coupled to the communication circuit for operating as an antenna.

18. The intrauterine device of claim 14, further comprising:
a copper component coupled to at least one of the body and the at least one arm for releasing copper into the uterus or a hormone component coupled to at least one of the body and the at least one arm for releasing a hormone into the uterus.

19. An intrauterine device comprising:
a body including a first end and a second end;
at least one arm extending radially from the first end when the body is within a uterus; and
a removal mechanism with a magnetic portion coupled to the second end for removing the intrauterine device from the uterus.

20. The intrauterine device of claim 19, wherein the removal mechanism is further for magnetically docking with a removal tool, wherein the removal mechanism includes a first portion and a second portion and the second portion is farther from the body than the first portion and the second portion has a greater circumference than the first portion for locking to the removal tool by when a loop attached to the removal tool is tightened around the first portion such that the loop has a smaller diameter than the second portion, the intrauterine device further comprising a communication circuit coupled to at least one of the body and the at least one arm for responding to a first signal by transmitting a second signal.

21. The intrauterine device of claim 20, further comprising:
memory communicatively coupled to the communication circuit for storing data, and wherein the second signal includes at least a portion of the data, wherein the portion of the data comprises at least one of a manufacturer of the intrauterine device, a date of production of the intrauterine device, a physician that performed an insertion of the intrauterine device, a date of insertion of the intrauterine device, and a type of the intrauterine device; and
one or more sensors communicatively coupled to the communication circuit for measuring health data associated with a user of the intrauterine device, and wherein the second signal includes at least a portion of the health data, wherein a sensor of the one or more sensors is exposable to bodily fluids for measuring at least one of glucose level of the user and iron level of the user, wherein the communication circuit is a near field communication chip and at least one of the communication circuit and at least one sensor of the one or more sensors are powerable by the first signal,
wherein the communication circuit is further for modulating the second signal to indicate the magnetic portion is docked with a removal tool.

22. The intrauterine device of claim 19, wherein the at least one arm in a first position is directly coupled to the body and in a second position is detached from the body, and the intrauterine device further comprises at least one connector for linking the at least one arm to the body such that the at least one arm remains linked to the body in both the first position and the second position via the at least one connector.

23. The intrauterine device of claim 19, further comprising:
a copper component coupled to at least one of the body and the at least one arm for releasing copper into the uterus or a hormone component coupled to at least one of the body and the at least one arm for releasing a hormone into the uterus.

24. A method comprising:
inserting a removal tool including a magnetic end through a cervix and into a uterus;
docking the magnetic end with a magnetic portion of a removal mechanism of an intrauterine device within the uterus; and
removing the intrauterine device from the uterus with the removal tool.

25. The method of claim 24, wherein the removal mechanism includes a first portion and a second portion and the second portion is farther from a body of the intrauterine device than the first portion and has a greater circumference than the first portion, wherein docking includes tightening a loop attached to the removal tool around the first portion such that the loop has a smaller diameter than the second portion.

26. The method of claim 24, wherein docking the removal tool further comprises activating an indicator light for indicating the removal tool has coupled with the magnetic portion.

27. The method of claim 24, wherein the intrauterine device further comprises a communication circuit for responding to a first signal by transmitting a second signal, wherein the second signal is modulated to indicate that the removal tool is docked to the removal mechanism, wherein transmitting the first signal to the communication circuit further comprises transmitting the first signal to a copper component that operates as an antenna and is communicatively coupled to the communication circuit.
